(19) United States
(12) Patent Application Publication
(10) Pub. No.: US 2025/0339066 A1
(43) Pub. Date: Nov. 6, 2025

(54) BLOOD DRAWING DEVICE

(71) Applicant: VITAL BIOSCIENCES INC., Mississauga (CA)

(72) Inventors: Srinivas Nadella, Mississauga (CA); Iman Khodadadadzadeh, Mississauga (CA)

(21) Appl. No.: 18/855,553

(22) PCT Filed: Jun. 9, 2023
(86) PCT No.: PCT/IB2023/000358

§ 371 Date: Oct. 9, 2024

Related U.S. Application Data
(60) Provisional application No. 63/350,704, filed on Jun. 9, 2022.

(51) Int. Cl. A61B 5/15; A61B 5/151

(52) U.S. Cl. CPC A61B 5/150076; A61B 5/150022; A61B 5/150099; A61B 5/15142

(57) ABSTRACT

A blood drawing device for collecting a blood sample from a patient. The blood drawing device includes a disposable collection cartridge and a reusable actuation device releasably coupled to the disposable collection cartridge. The disposable collection cartridge includes a housing, a puncture element and a fluid container. The reusable actuation device includes a controller, a power source and a vacuum source.

### CROSS-REFERENCE TO RELATED APPLICATIONS

This application claims the benefit of U.S. Provisional Patent Application No. 63/350,704 filed Jun. 9, 2022 entitled “Blood Drawing Device”, which is incorporated by reference herein in its entirety.

### TECHNICAL FIELD

The present disclosure generally relates to a blood drawing device for collecting a blood sample from a patient and methods of using the same.

### SUMMARY

Disclosed herein is a device for collecting a blood sample from a patient. In an embodiment, the blood drawing devices may include a disposable collection cartridge and a reusable actuation device releasably coupled to the disposable collection cartridge. The disposable collection cartridge may include a housing, a puncture element and a fluid container. The reusable actuation device may include a controller in electronic communication with the disposable collection cartridge, a power source in electronic communication with the disposable collection cartridge and a vacuum source in fluid communication with the disposable collection cartridge.

In an embodiment, the disposable collection cartridge may further include a heating element coupled to a surface of the housing extending from the reusable actuation device. The heating element may include a flexible printed circuit board (PCB) and a compliant layer. The heating element may include an aperture, and the puncture element may be movable relative to the housing between a retracted position and an extended position. The puncture element may be contained completely within the housing in the retracted position, and the puncture element may extend through the aperture of the heating element in the extended position. The heating element may be planar and have a circular portion surrounding the aperture and a tab extending radially from the circular portion. The tab may include at least one exposed electrical contact that electrically couples the heating element to the reusable actuation device. In an embodiment, the disposable collection cartridge may include an adhesive tape, and an aperture of the disposable collection cartridge may include an anticoagulation film.

In an embodiment, the reusable actuation device may further comprise a controller. The controller may be configured to control the vacuum source, wherein the vacuum source is a vacuum pump; a mechanical actuation system configured to actuate the puncture element; a temperature sensor coupled to a heating element; a pressure sensor disposed within the disposable collection cartridge; and a sample detection sensor disposed within the fluid container. The temperature sensor may be coupled to the controller, and the controller may be configured to adjust a temperature of the heating element based on a temperature detected by the temperature sensor and a desired temperature. The reusable actuation device may comprise an adjustable knob coupled to the controller and configured to control the desired temperature of the heating element.

In an embodiment, the vacuum source may be a vacuum pump configured to provide vacuum proximate a target area while the puncture element is in a retracted position and during movement of the puncture element into an extended position. The vacuum pump may be configured to maintain a predetermined profile of pressure in the disposable collection cartridge during collection of the blood sample. In some embodiments, the vacuum pump may be configured to apply one or more variable vacuum profiles in the disposable collection cartridge during collection of the blood sample.

In an embodiment, the fluid container may include a capillary channel in fluid communication with an open end of the fluid container. In some embodiment, the capillary channel may include an anticoagulation agent. In some embodiments, the fluid container may include a lip extending axially from an end portion thereof. In some embodiments, the blood drawing device may further include a fluid reservoir, the fluid reservoir comprising a liquid reagent. In some embodiments, the liquid reagent may treat the blood during collection of the blood sample. The liquid reagent may comprise at least one of lithium heparin, K2-EDTA, K3-EDTA, trisodium citrate or another acceptable anticoagulant reagent.

In an embodiment, the puncture element may include one or more lancets. In some embodiments, the vacuum source may be a vacuum pump configured to continuously pump air out of the disposable collection cartridge before and during collection of the blood sample. In some embodiments, the fluid container may be coupled to the disposable collection cartridge by a threaded coupling. In some embodiments, the fluid container may be positioned at an oblique angle relative to the puncture element when the disposable collection cartridge is coupled with the reusable actuation device.

### BRIEF DESCRIPTION OF THE DRAWINGS

The following detailed description of embodiments of the blood drawing device, will be better understood when read in conjunction with the appended drawings of an exemplary embodiment. It should be understood, however, that the invention is not limited to the precise arrangements and instrumentalities shown.

In the drawings:

FIG. 1 is a front perspective view of a blood drawing device in accordance with an exemplary embodiment of the present disclosure;

FIG. 2 is a rear perspective view of the blood drawing device of FIG. 1;

FIG. 3 is a perspective view of the disposable collection cartridge of the blood drawing device shown in FIG. 1;

FIG. 4 is a side cross sectional view of the disposable collection cartridge of FIG. 3;

FIG. 5 is a rear perspective cross sectional view of the disposable collection cartridge of FIG. 3;

FIG. 6 is a side cross sectional view of the disposable collection cartridge of FIG. 3 showing the piercing element in the extended position;

FIG. 7 is a font view of a heating element of the blood drawing device of FIG. 1;

FIG. 8 is a perspective partial cross sectional view of the blood drawing device shown in FIG. 1;

FIG. 9 is a magnified side cross sectional view of the blood drawing of FIG. 1 shown with an adhesive tape on the compliant layer;

FIG. 10 is a perspective view of a first exemplary lancet for use in the blood drawing device shown in FIG. 1; and

FIG. 11 is a perspective view of a second exemplary lancet for use in the blood drawing device shown in FIG. 1.

### DETAILED DESCRIPTION

Blood sampling and analysis are indispensable parts of a patient's diagnostics. Blood quality is the metric that is of utmost importance in clinical chemistry/pathology. Traditional methods of blood extraction are based on decades-old technologies such as the venipuncture (phlebotomy). But the phlebotomy process can be traumatic and inconvenient for some patients. Some approaches, such as a finger prick (using a lancet), allow drawing blood without the need for phlebotomy. This method is the most common method for checking blood glucose levels. For neonates, a heal prick is used to extract small blood sample for a select few screening tests. The chief shortcoming of these methods is that the volume of blood extracted is limited by the amount of blood available in the capillary blood vessels that have been severed as a result of the lancing process, before the repair process is initiated by the body. Repeated squeezing (milking) can be used to slightly increase the volume of expelled blood, but it is quite uncomfortable and laborious.

Some existing approaches to collecting capillary blood, as opposed to venous blood, allow collecting larger volumes of blood. Thus, some approaches allow creating several puncture wounds for collecting about 200 uL of blood from capillaries after several minutes of use. However, one of the concerns with testing capillary blood is the fact that this method of extraction of blood has adverse effects on some blood parameters, which would then result in mis-diagnosis of a patient. The parameters that are most susceptible are white blood cells (WBC) count, red blood cells (RBC) count, platelet count, and potassium, more generally complete blood count (CBC) and electrolyte panels. The CBC and electrolyte panels are two of the most commonly requisitioned panels and these parameters are some of the most important parameters considered by physicians to determine the overall health of a patient. Thus, any deviation from the actual values can lead to misdiagnosis and therefore mistreatment of the patient.

Non-phlebotomy approaches to blood collection, as compared to phlebotomy-based approaches, are complicated due to the increase in WBC count (which can be caused by the body's response to managing the wound as well as potential clumping of platelets that are mistakenly counted as WBCs), decrease in RBC count (the destruction of these fragile cells via the hemolysis process as a result of shear forces while the blood is being forced through the flesh wound), decrease in platelet count (these cells are responsible for blood coagulation and they clump and attempt to stop the bleeding when they come in contact with air and also as a result of shear forces as the blood is being forced through the flesh wound), and increase in potassium concentration (a side effect of hemolysis as red cells include a large amount of potassium inside which is not indicative of the true concentration of potassium).

In general, capillary blood collection methods have not been able to address the above issues and therefore have limited clinical utility as a general-purpose blood extraction method. In addition to the blood quality issues, lancing the finger may be an uncomfortable and painful process as there are many nerve endings at the tip of fingers. The amount of blood available for collection is also limited, which means that the finger will have to be “milked” in order to increase the sample volume, which reduces the quality of the extracted blood.

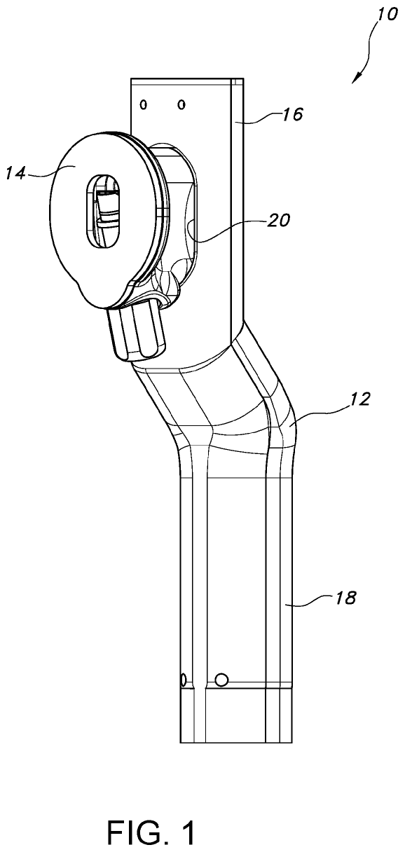

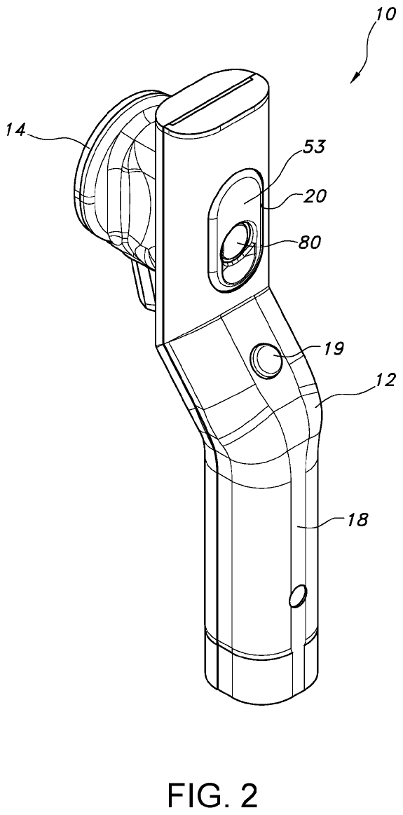

Referring to the drawings in detail, wherein like reference numerals indicate like elements throughout, there is shown in FIGS. 1-11 a blood drawing device, generally designated 10, in accordance with an exemplary embodiment of the present invention. The blood drawing device 10 may include two main components, a reusable actuation device 12 and a disposable collection cartridge 14. Because the reusable actuation device does not contact a patient's blood or blood sample, one reusable actuation device 12 may be used with two or more disposable collection cartridges 14. Before and after collection of the blood sample the disposable collection cartridge 14 may be separated 10 from the reusable actuation device 12. During collection of the blood sample, the disposable collection cartridge 14 may be coupled to the reusable actuation device 12, which may control the operation of the components in the disposable collection cartridge 14. By separating the blood drawing device 10 into multiple pieces, it may be possible to enhance the blood draw process by applying active control systems which may be configured to adjust the blood draw parameters based 15 on the patient's needs. By including the active elements in the reusable actuation device 12, it may be possible to reduce the waste and costs associated with including these features in traditional single-use blood drawing devices.

Referring to FIG. 1, the blood drawing device 10 may have a reusable actuation device 12 that is used in conjunction with a disposable collection cartridge 14 that is configured to capture the blood sample from the patient. The disposable collection cartridge 14 can be discarded after use. As discussed in more detail below, the disposable collection cartridge 14 may be used to puncture a patient's body and to collect a blood sample from the patient's body through the puncture, such that the collected blood is transferred into the disposable collection cartridge 14. FIG. 1 illustrates the blood drawing device 10 with the reusable actuation device 12 and disposable collection cartridge 14 separated.

As shown in FIG. 1, the reusable actuation device 12 may comprise a body 16 having a proximal end, and a handle 18 coupled to the proximal end of the body 16. The handle 18 may have a grip portion and other features that allow it to be conveniently held by a user. In some embodiments, the reusable actuation device 12 does not have a handle. The body 16 of the reusable actuation device 30 12 may have a cavity 20 configured to releasably receive the disposable collection cartridge 14 to couple to the reusable actuation device 12 and the disposable collection cartridge 14. The body 16 and handle 18 of the reusable actuation device 12 may be separated by a curvature. The curvature between the body 16 and handle 18 may be configured to extend the body 16 forward relative to the handle 18. This orientation of the reusable actuation device 12 may allow for a user to grasp the handle 18 without interfering with application of the disposable collection 14 in the body 16.

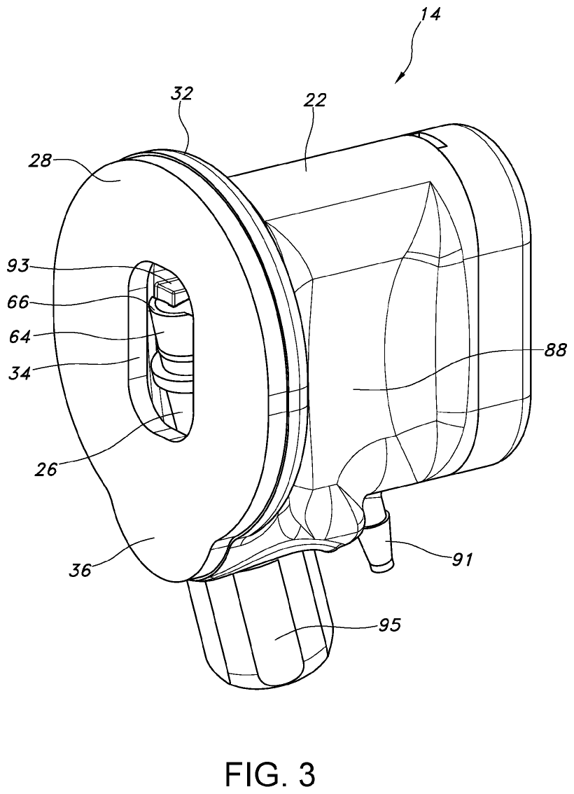

As shown in FIGS. 3-5, the disposable collection cartridge 14 in some embodiments comprises a housing 22, a puncture element 24 and a fluid container 26. The disposable collection cartridge 14 may further comprise a heating element 28 coupled to the disposable collection cartridge 14. In one embodiment, the surface containing the heating element 28 is the surface of the disposable collection cartridge 14 that is opposite the surface that couples to the reusable actuation device 12. The heating element 28 may apply heat proximate the target area of the patient's skin before and during collection of the blood sample. By applying heat proximate the target area, the thermal energy may cause the blood in that area of the skin to flow more rapidly, which may allow for more blood to be collected. The heating element 28 may apply a constant heat throughout collection of the sample. In some embodiments, the heating element 28 may apply variable heat according to a predetermined interval.

The heating element may be configured to apply a temperature between 30-45 degrees Celsius proximate the target area. The heating element may be configured to apply a temperature of approximately 25 degrees Celsius proximate the target area. The heating element may be configured to apply a temperature of approximately 30 degrees Celsius proximate the target area. The heating element may be configured to apply a temperature of approximately 35 degrees Celsius proximate the target area. The heating element may be configured to apply a temperature of approximately 40 degrees Celsius proximate the target area. The heating element may be configured to apply a temperature of approximately 45 degrees Celsius proximate the target area. The heating element may be configured to apply a temperature of approximately 50 degrees Celsius proximate the target area.

The disposable collection cartridge 14 may further include at least one fastening element 88 configured to releasably couple to the reusable actuation device. The fastening element 88 may be configured to be received in the cavity 20. In some embodiments, the fastening element 88 may be a magnet. The reusable actuation device 12 may include alignment features to ensure the disposable collection cartridge 14 is properly oriented in the cavity 20. There may be an indication that the disposable collection cartridge 14 has been properly coupled to the reusable actuation device 12. For example, there may be an audible or visual confirmation. A light on the reusable actuation device 12 may turn on when the disposable collection cartridge 14 is properly oriented in the cavity 20. In some embodiments, a click or other noise may confirm that the disposable collection cartridge 14 is properly oriented in the cavity 20.

In some embodiments, the heating element 28 includes a flexible printed circuit board (PCB) 30. The heating element 28 may include a disposable restrictive circuit on the flexible PCB 30. The heating element 28 may include a compliant layer 32. The compliant layer 32 may provide a cushion to soften the surface that contacts the patient's skin. The compliant layer 32 may also distribute the heat produced by the heating element 28 to improve the temperature uniformity proximate the target area. In some embodiments, the compliant layer 32 is a closed cell foam. In some embodiments, the compliant layer 32 is approximately 3 mm in thickness. In some embodiments, the compliant layer 32 is less than 5 mm in thickness. In some embodiments, the compliant layer 32 is less than 3 mm in thickness. In some embodiments, the compliant layer 32 is between 0.5 mm and 3 mm in thickness. In alternative embodiments, the heating element 28 could include a non-compliant layer for distributing the heat and contacting the skin surface.

The flexible PCB 30 may be embedded in the compliant layer 32. In other embodiments, the flexible PCB 30 could be coupled to the rear surface of the compliant layer 32. The flexible PCB 30 may include a heat conductor 33. The heat conductor 33 may be made from on one of copper traces, etched foil, or wire. The heat conductor 33 may be a single piece of material extending in an organized configuration to cover the area of the heating element 28. Example layouts of the heat conductor 33 may include any or all of a serpentine layout, a double square spiral, a peripheral modified spiral, a S shape, parallel-lines or a lens shape. As electricity is delivered to the heat conductor, some form of resistance may be used to create heat. Beyond the resistance, the amount of heat supplied to a patient by the heat conductor 33 may depend on multiple factors, such as the material the heat conductor 33 is made from, the thickness of the compliant layer 32, and the amount of electrical input delivered to the heat conductor 33.

A thickness of the heating element 28 may be less than the thickness of the compliant layer 32 to allow the heating element 28 to be disposed within the compliant layer 32. In some embodiments, the heating element 28 is approximately 2 mm in thickness. In some embodiments, the heating element 28 is less than 4 mm in thickness. In some embodiments, the heating element 28 is less than 2 mm in thickness. In some embodiments, the heating element 28 is between 0.1 mm and 3 mm in thickness.

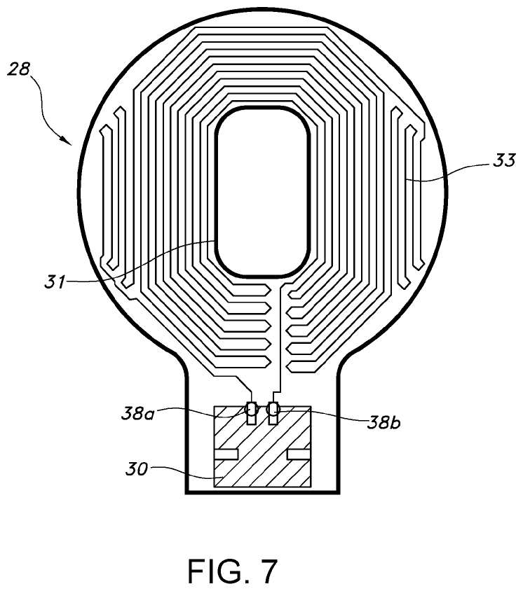

As shown in FIG. 7, in some embodiments, the heating element 28 is planar. The heating element 28 may have a generally circular portion surrounding the aperture 34 and a tab 36 extending radially from the circular portion. To avoid interfering with the movement of the puncture element 24, heating element 28 may include an opening 31. The opening 31 may be shaped and sized to fit around aperture 34. The opening 31 may be substantially the same size as aperture 34. In some embodiments, the opening 31 is larger than aperture 34. In some embodiments, the heating element 28 may have a generally square shape surrounding the aperture 34. The tab 36 may be shaped and sized to achieve the desired application, for example generally circular, as shown in FIG. 3, or generally rectangular, as shown in FIG. 7. The tab 36 may include at least one exposed electrical contact 38a, 38b which may interact with an electrical contact 41 (not shown) disposed on the housing 22 to supply the heating element 28 with power.

The electrical contacts 38a, 38b may be configured to electrically couple the heating element 28 to the reusable actuation device 12 when the disposable collection cartridge 14 is received in the cavity 20. Electrical contact 41 may include more than one electrical contact. The housing may include an electrical contact 41 on the surface to which the heating element 28 is coupled. Electrical contact 41 of the housing 22 may be located adjacent to the electrical contacts 38a, 38b of the heating element 28 when the heating element 28 is coupled to the housing 22. The electrical contact 41 of the housing 22 may run within the housing 22 from the surface adjacent the heating element 28 to a surface of the housing 22 that contacts the reusable actuation device 12 when the disposable collection cartridge 14 is received in the cavity 20. The housing 22 may include an electrical contact 43 on a portion the surface that contacts the reusable actuation device 12. Electrical contact 43 may include more than one electrical contact. The reusable actuation device 12 may include the electrical contact 45 on the surface adjacent to electrical contact 43 of the housing. Electrical contact 45 may include more than one electrical contact. Electrical contact 45 of the reusable actuation device 12 may be electrically coupled to the power source 48 by a wired connection. While electrical contacts are described above as providing power from the power source 48 to the heating element 28, any acceptable form of electrical communication may be provided. For example, wireless power transfer or electromagnetic power transfer.

In some embodiments, the heating element 28 is positioned along a plane that is perpendicular to the path of the puncture element 24. As shown in FIG. 7, the heating element 28 may include an opening 31. In some embodiments, the heating element 28 covers the entire surface of the disposable collection cartridge 14 that surrounds the aperture 34. In some embodiments, the heating element 28 covers only a portion of the surface of the disposable collection cartridge 14 that surrounds the aperture 34.

In some embodiments, the temperature sensor 54 is electrically coupled to the controller 46. The controller 46 may be configured to adjust the temperature of the heating element 28 based on a difference between a temperature detected by the temperature sensor 54 and a desired temperature. In some embodiments, the temperature sensor 54 provides feedback to the controller 46 as a means to perform active temperature control throughout the blood draw process to maintain the provided temperature within a predetermined tolerable range. In some embodiments, it is necessary and useful, to implement different temperature profiles within the system to optimize blood flow throughout the blood draw process. The reusable actuation device 12 may include an adjustable knob 60 coupled to the controller 46. In some embodiments, the user can manipulate the adjustable knob 60 to control a temperature of the heating element 28. In some embodiments, the temperature sensor 54 detects a user's heat profile and provides feedback to the controller 46 in order to activate the vacuum source 50 as a result of the disposable collection cartridge 14 being pressed against the patient's skin.

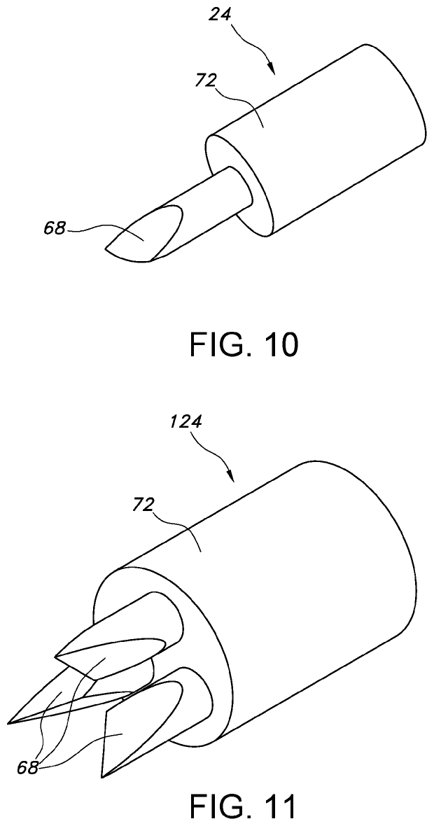

As described below in connection with FIGS. 10-11, the puncture element 24 may contain one or more piercing elements 68 (e.g., lancets or needles) fixed thereto to pierce a patient's skin and facilitate a blood draw, as described in more detail below. The puncture element 24 may be movable relative to the housing to move the piercing element(s) into the patient's skin. The puncture element 24 may have a retracted position and an extended position. In some embodiments, the puncture element 24 is contained completely within the housing in the retracted position. In some embodiments, the puncture element extends through the aperture 34 of the heating element 28 in the extended position. The puncture element 24 may move along a path that is generally perpendicular with the body 16 of the reusable actuation device 12.

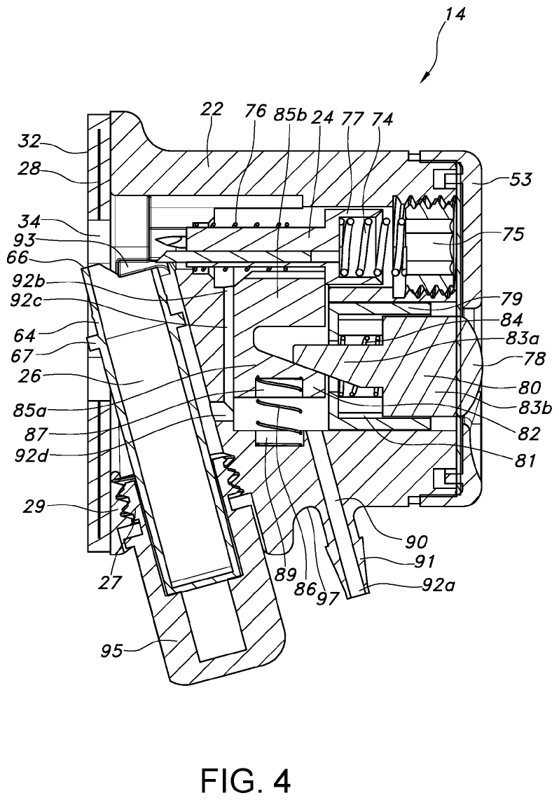

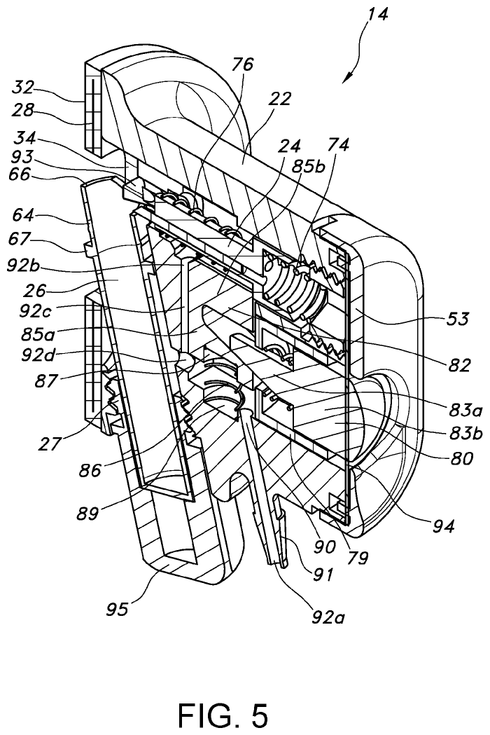

As shown in FIGS. 4-5, in some embodiments, the puncture element 24 is powered by a first biasing element 74 that drives the puncture element 24 from the retracted position to the extended position. The first biasing element 74 may be threadedly coupled to the housing 22 by an anchor 75 at a first side. The anchor 75 may vary in size and shape depending on the size and shape requirements of the first biasing element 74. Anchor 75 may prevent the first side of the first biasing element 74 from moving relative to the housing when the puncture element is in the extended position. The first biasing element 74 may be received within a position of the puncture element at a second side. The puncture element 24 may include a recess 77 configured to receive the second side of the first biasing element 74. The first biasing element 74 may be a spring. In some embodiments, the puncture element 24 is returned from the extracted position to the retracted position by a second biasing element 76. The second biasing element 76 may be a spring. In some embodiments, the first biasing element 74 generates a greater force than the second biasing element 76. The disposable collection cartridge 14 may serve as a sharps containment device after the puncture element 24 has returned to the retracted position. The disposable collection cartridge 14 may shroud the puncture element 24 after use.

The second biasing element 76 may provide a biasing force sufficient to allow the puncture element 24 to extend approximately 3 mm out of the disposable collection cartridge 14 before returning it to the retracted position. The second biasing element 76 may provide a biasing force sufficient to allow the puncture element 24 to extend approximately 2.5 mm out of the disposable collection cartridge 14 before returning it to the retracted position. The second biasing element 76 may provide a biasing force sufficient to allow the puncture element 24 to extend approximately 3.5 mm out of the disposable collection cartridge 14 before returning it to the retracted position.

As shown in FIGS. 4-5, the puncture element may be actuated by a firing mechanism 78 located within the disposable collection cartridge 14. The firing mechanism 78 may be controlled by the user. In some embodiments, the firing mechanism is located within the reusable actuation device. In some embodiments, the firing mechanism 78 consists of an actuation button 80 and a release element 82. In a locked position, the release element 82 may be positioned to prevent the puncture element 24 from moving from the retracted position to the extended position.

In some embodiments, the actuation button 80 has a proximal end and a distal end, as shown in FIGS. 4-5. The proximal end of the actuation button 80 may be biased by the user to actuate the firing mechanism 78. The distal end 83 of the actuation button 80 may extend into the housing 22 and interact with the release element 82. The distal end 83a may have a thickness that is less than that of the proximal end 83b of the actuation button 80 and may be angled or slanted as to be received by a portion of the release element 82. The actuation button 80 may be urged toward the proximal end 83b by a third biasing element 84 to prevent unintended actuation of the firing mechanism 78. The third biasing element 84 may be a spring. A rear housing 53 coupled to the housing 22 may block and prevent the third biasing element 84 from moving the actuation button 80 beyond the housing 22. In some embodiments, rear housing 53 may be removable to allow the first biasing element 74 and/or the puncture element 24 to be replaced.

A button guide 79 may be included around the actuation button 80 within the housing, as shown in FIGS. 4-5. The button guide 79 may be sized to allow the actuation button 80 to slide therein but prevent the actuation button 80 from unintended rotation or movements. The button guide 79 may include a track 81 extending therefrom which engages the actuation button 80 to prevent the actuation button 80 from rotating relative to the housing 22 when biased by the user.

In some embodiments, the release element 82 has a proximal end 85a and a distal end 85b, as shown in FIGS. 4-5. The distal end 85b of the release element 82 may be configured to engage the puncture element 24 to prevent movement before the actuation button 80 is moved. The proximal end 85a of the release element 82 may be shaped to receive the distal end 83a of the actuation button 80. The shape of the proximal end 85a of the release element 82 may be about the same angle as that of the distal end 83a of the actuation button 80.

The release element 82 may be urged into the locked position by a fourth biasing element 86, as shown in FIGS. 4-5. The fourth biasing element 86 may be a spring. The proximal end 85a of the release element 82 may include a catch 87 configured to securely receive the fourth biasing element 86. The catch 87 may be generally cylindrical and have a circumference similar to that of the fourth biasing element 86. The housing 22 may include a catch 89 configured to securely receive the fourth biasing element 86. The catch 89 may be generally cylindrical and have a circumference similar to that of the fourth biasing element 86.

When a user presses the proximal end of the actuation button 80, the release element 82 may be urged by the distal end of the actuation button 80 toward the proximal end of the release element 82. When the release element 82 is displaced proximally, the puncture element 24 may be unrestricted by the distal end 85b of the release element 82 and may move from the retracted position to the extended position.

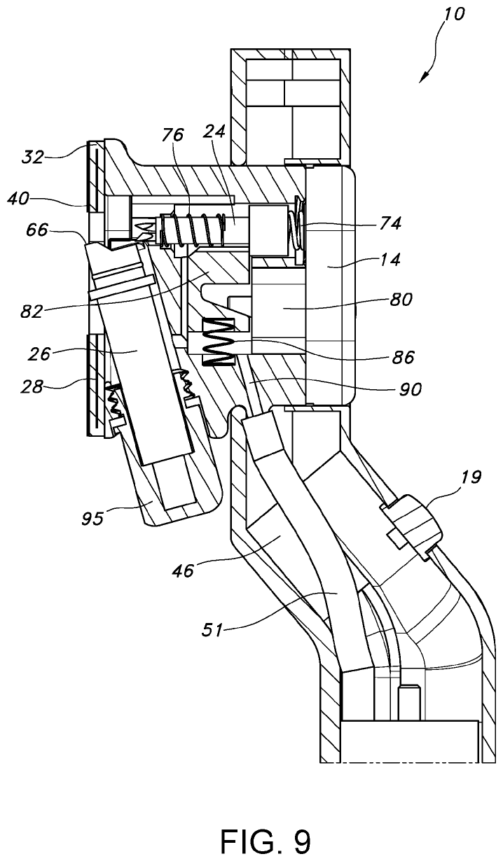

Reducing movement of the disposable collection cartridge 14 may improve the performance of the blood drawing device 10 by ensuring repeatability. As shown in FIG. 9, in some embodiments, the disposable collection cartridge 14 includes an adhesive tape 40 on an outer facing surface of the compliant layer 32. The adhesive tape 40 may prevent the disposable collection cartridge 14 from moving relative to the patient's skin during collection of a sample. The adhesive tape 40 may include a release liner (e.g., a plastic film) (not shown) covering the adhesive tape 40 until use to reduce unintended adhesion. The release liner may cover the aperture 34 to prevent contamination of the components of the disposable collection cartridge 14 prior to use. In some embodiments, the release liner may cover only the adhesive tape 40. The adhesive tape 40 may form an airtight seal between the disposable collection cartridge 14 and the patient when the disposable collection cartridge 14 is pressed against the patient's skin. In some embodiments, the aperture 34 includes an anticoagulation film 44 that remains in contact with the user's skin throughout the blood draw process. The anticoagulation film 44 may cover the entire opening defined by the aperture 34. In some embodiments, the anticoagulation film 44 may cover only a portion of the opening defined by the aperture 34.

Increasing pressure proximate the target area may improve the patient's experience and facilitate a higher volume and faster blood draw than would be possible without increasing the pressure. Further, to promote blood flow without causing pain or damaging the target area, it is important to precisely control the amount of pressure being applied. As such, a vacuum may modulate the amount of pressure proximate the target area, as described in more detail below. A vacuum may further reduce the amount of lancing and repeated squeezing required to collect the desired amount of blood.

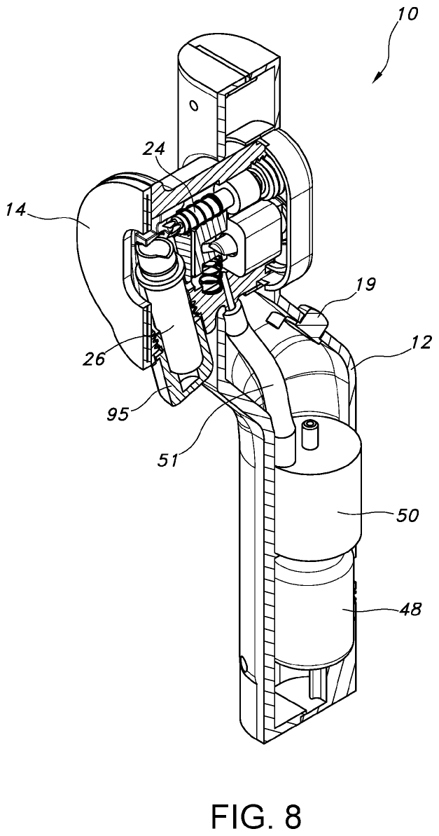

As shown in FIGS. 8-9, the reusable actuation device 12 may comprise a controller 46 in electronic communication with the disposable collection cartridge 14, a power source 48 in electronic communication with the disposable collection cartridge 14, and a vacuum source 50 in fluid communication with the disposable collection cartridge 14 via a vacuum duct 51. In some embodiments, the vacuum duct 51 is a pneumatic tube, pipe, or other conduit. The reusable actuation device 12 may be powered by a battery. In some embodiments, the reusable actuation device 12 is powered by a power cord plugged into an energy source.

In some embodiments, the controller 46 is configured to control the vacuum source 50. In some embodiments, the vacuum source 50 is a vacuum pump. In some embodiments, the controller 46 is configured to control a mechanical actuation system 52 that actuates the puncture element 24 when the disposable collection cartridge 14 is received in the cavity 20. In some embodiments, the controller 46 is configured to control a temperature sensor 54 coupled to the heating element 28 when the disposable collection cartridge 14 is received in the cavity 20. In some embodiments, the controller 46 is configured to control a pressure sensor 56 disposed within the housing 22 when the disposable collection cartridge 14 is received in the cavity 20. In some embodiments, the pressure sensor 56 is disposed within the reusable actuation device 12. The pressure sensor 56 may be disposed within the vacuum duct 51 of the reusable actuation device 12.

In some embodiments, the controller 46 is configured to control a sample detection sensor 58 disposed within the fluid container 26 when the disposable collection cartridge 14 is received in the cavity 20. The fluid container 26 may be any container with an internal space that is configured to hold a collected fluid. The fluid container 26 may also be referred to as a collection tube. In some embodiments, the sample detection sensor 58 is disposed within the reusable actuation device 12. If the sample detection sensor 58 is disposed within the reusable actuation device 12, the sample detection sensor 58 may detect the sample through, for example, a transparent window of the fluid container 26.

In some embodiments, the controller 46 may be implemented in the form of hardware, may be implemented in the form of software, or may be implemented in the form of a combination thereof. In some embodiments, the controller 46 according to an exemplary embodiment of the present disclosure may be a processor (e.g., computer, microprocessor, CPU, ASIC, circuitry, logic circuits, etc.). The processor may be implemented by a non-transitory memory storing, e.g., a program(s), software instructions reproducing algorithms, etc., which, when executed, may perform the overall control of the vacuum source 50, the mechanical actuation system 52, the temperature sensor 54, the pressure sensor, and the sample detection sensor 58, and a processor configured to execute the program(s), software instructions reproducing algorithms, etc. In some embodiments, the memory and the processor may be implemented as separate semiconductor circuits. Alternatively, the memory and the processor may be implemented as a single integrated semiconductor circuit. In some embodiments, the processor may embody one or more processor.

In some embodiments, the vacuum source 50 is a vacuum pump configured to provide a vacuum in the housing 22 proximate a target area while the puncture element 24 is in the retracted position. The vacuum source 50 may be a vacuum pump configured to provide a vacuum in the housing 22 proximate a target area while the puncture element 24 is in the extended position. The pressure sensor 56 may provide feedback to the controller 46 as a means to perform active pressure control throughout the blood draw process.

As described in more detail below, active control of the vacuum source 50 may increase the accuracy and efficiency of blood collection. Pressure created by the vacuum source 50 in the housing 22 may be increased to facilitate greater blood flow from the target area. Pressure created by the vacuum source 50 in the housing 22 may be decreased to facilitate lesser blood flow from the target area. In some embodiments, the vacuum source 50 allows for between a 3 psi and 10 psi draw. In some embodiments, the vacuum source 50 allows for up to a 2 psi draw. In some embodiments, the vacuum source 50 allows for up to a 3 psi draw. In some embodiments, the vacuum source 50 allows for up to a 4 psi draw. In some embodiments, the vacuum source 50 allows for up to a 5 psi draw. In some embodiments, the vacuum source 50 allows for up to a 6 psi draw. In some embodiments, the vacuum source 50 allows for up to a 7 psi draw. In some embodiments, the vacuum source 50 allows for up to an 8 psi draw. In some embodiments, the vacuum source 50 allows for up to a 9 psi draw. In some embodiments, the vacuum source 50 allows for up to a 10 psi draw. In some embodiments, the vacuum source 50 allows for up to an 11 psi draw.

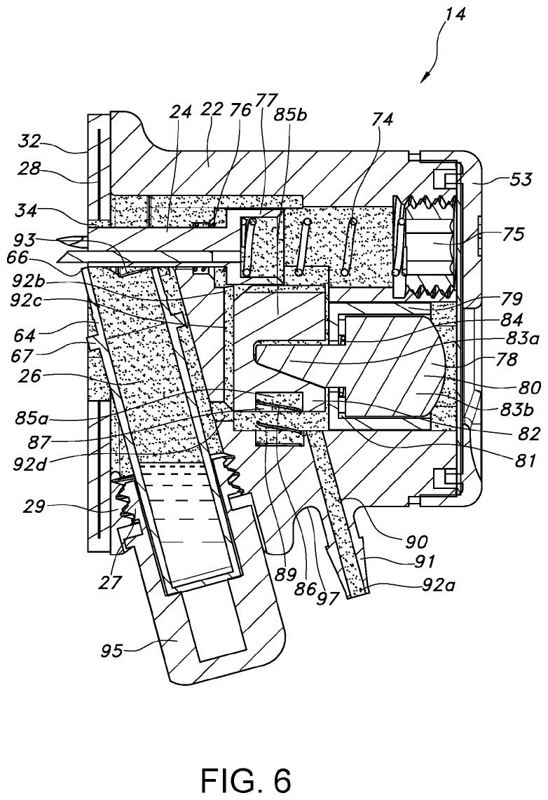

The vacuum source 50 in the reusable actuation device 12 may be in fluid communication with a vacuum channel 90 in the disposable collection cartridge 14. As shown in FIGS. 5-7, the vacuum channel 90 may extend from an exterior side 92a to an interior side 92b. Vacuum channel 90 may form a fluid path between the target area and the vacuum source 50 to allow the vacuum source 50 to control the pressure proximate the target area. From the exterior side 92a the vacuum channel 90 may extend through the housing 22 to an area proximate the firing mechanism 78. The vacuum channel 90 may then extend through separate portions of the housing 22 to an opening 92c proximate the puncture element 24 and an opening 92d proximate the fluid container 26. The flow path of the vacuum channel 90 is shown in broken line for reference in FIG. 4. In some embodiments, the vacuum channel 90 may include all open areas within the housing 22, including but not limited to, all areas around the firing mechanism 78, the fluid container 26 and the puncture element 24, as shown with stippling in FIG. 6.

The vacuum source 50 may be fluidly coupled to the disposable collection cartridge 14. The exterior side 92a may engage the vacuum source, and the interior side 92b may direct the vacuum draw to a location proximate the target area. The exterior side 92a may include a tip 91 protruding from housing 22. Tip 91 may be tapered to facilitate a connection between the vacuum channel 90 with the vacuum duct 51 when the disposable collection cartridge 14 is received in the cavity 20. The shape and size of the tip 91 may form an airtight seal with the vacuum duct 51 when the disposable collection cartridge 14 is received in the cavity 20. In some embodiments, the tip 91 includes a sheath to ensure the shape and size of the tip 91 forms an airtight seal with the vacuum duct 51 when the disposable collection cartridge 14 is received in the cavity 20.

Referring to FIG. 4, tip 91 may need to be inserted into the reusable actuation device 12 first because it protrudes from the housing 22 of the disposable collection cartridge 14. As such, housing 22 may include a cutout proximate the tip 91 to receive a portion of the reusable actuation device 12 to facilitate insertion of the tip 91 into the reusable actuation device 12. Once the tip 91 is inserted into the reusable actuation device 12, the disposable collection cartridge 14 may be pivoted about the cutout 97 until the disposable collection cartridge 14 is completely received in the cavity 20 of the reusable actuation device 12. The cutout 97 may be generally rounded to promote the disposable collection cartridge 14 to rotate relative to the reusable actuation device 12 when disposed therein.

In some embodiments, the vacuum source 50 is configured to maintain a predetermined profile of pressure in the disposable collection cartridge 14 during collection of the blood sample. For example, a constant pressure (e.g., 7 psi) may be applied by the vacuum source 50 during collection of the blood sample to draw the sample out of the patient and into the fluid container 26. In some embodiments, the vacuum source 50 is configured to apply one or more variable vacuum profiles in the disposable collection cartridge 14 during collection of the blood sample. For example, the pressure applied by the vacuum source 50 may repeatedly increase for a set amount of time (e.g., 5 seconds) and then decrease for a set amount of time (e.g., 5 seconds) during collection of the blood sample. The vacuum source 50 may be configured to continuously pump air out of the disposable collection cartridge 14 before collection of the blood sample. The vacuum source 50 may be configured to continuously pump air out of the disposable collection cartridge 14 during collection of the blood sample. When air is being constantly and continuously pumped out of the disposable collection cartridge 14, the internal pressure of the housing 22 may not rise over time and may allow for most consistent results. The controller 46 may adjust the pressure sensor 56 during collection of a blood sample depending on the feedback the controller receives from the sample detection sensor 58 (e.g., rate of blood flow).

Treatment of the blood during the blood collection process may further enhance the accuracy of test results. This treatment may include a reagent interacting with the blood at the blood collection site, while the blood is traveling between the blood collection site to an open end 64 of the fluid container 26, or while the blood is in the fluid container 26. In some embodiments, the fluid container 26 includes a capillary channel 62 (not shown) in fluid communication with the open end 64 of the fluid container 26. The capillary channel 62 may be referred to as a capillary tube. The housing 22 may include a ledge 93 extending from a portion thereof. The ledge 93 may be located near the open end 64 to orient the fluid capture element relative to the housing 22 during use. The ledge 93 may prevent the open end 64 from rotating relative to the housing 22 during use. In some embodiments, the open end 64 is cone shaped. In some embodiments, the capillary channel 62 includes an anticoagulation agent. This anticoagulation agent may be critical to preserving blood quality, especially for platelets and potassium. In some embodiments, more than one capillary channel 62 may be used to route the blood sample to more than one fluid container 26. The open end 64 may include a lip 66 extending from an end portion therefrom and include sidewalls treated with proper anticoagulant reagents in a dried format. The lip 66 may extend beyond the compliant layer 32 as to contact the user during the blood collection process.

In some embodiments, the disposable collection cartridge 14 includes a fluid reservoir 65 disposed within the fluid container 26. The fluid reservoir may be separate from but in fluid communication with the fluid container 26. The fluid reservoir 65 may be located proximate the lip 66, as shown in FIG. 3. The fluid reservoir 65 may contain a liquid reagent. During the blood collection process, a volume of the liquid reagent may be displaced from the fluid reservoir to treat the blood as it is collected during the blood collection process. In some embodiments, the liquid reagent is applied directly to the collection site to treat the blood as it is collected during the blood collection process. The liquid reagent may be one of lithium heparin, dipotassium ethylenediaminetetraacetic acid (K2-EDTA), tripotassium ethylenediaminetetraacetic acid (K3-EDTA), trisodium citrate or another acceptable testing reagent necessary for a specific testing to be performed on the collected blood.

In some embodiments, the fluid container 26 is coupled to the disposable collection cartridge 14 by a threaded coupling with an end retention cap 95, as shown in FIGS. 4-5. In some embodiments, the fluid container 26 is coupled directly to the housing 22 without the end retention cap 95. The end retention cap 95 may include a threaded collar 27 at one end thereof. Housing 22 may include a threaded receiver 29 shaped and sized to receive the threaded collar 27. Rotation of the end retention cap 95 in a first direction relative to the housing 22 may secure the fluid container 26 to the housing 22. Rotation of the end retention cap 95 is a second direction that is opposite the first direction relative to the housing 22 may release the fluid container 26 from the housing 22.

A plug (not shown) may be disposed in the fluid container 26 to prevent the sample from escaping when the fluid container 26 is separated from the housing 22. As discussed in more detail below, the open end 64 of the fluid container 26 may receive a cap to prevent the sample from escaping the fluid container 26 when the fluid container 26 is separated from the housing 22. In some embodiments, the fluid container 26 is at an oblique angle relative to the puncture element 24. A portion of the fluid container 26 may extend from the housing 22. In some embodiments, the fluid container 26 is contained entirely within the housing 22.

In some embodiments, the fluid container 26 holds between 10 uL and 1 mL of blood. The fluid container 26 may be configured to hold up to 1 mL of blood. The fluid container 26 may be configured to hold up to 900 uL of blood. The fluid container 26 may be configured to hold up to 800 uL of blood. The fluid container 26 may be configured to hold up to 700 uL of blood. The fluid container 26 may be configured to hold up to 600 uL of blood. The fluid container 26 may be configured to hold up to 500 uL of blood. The fluid container 26 may be configured to hold up to 400 uL of blood. The fluid container 26 may be configured to hold up to 350 uL of blood. The fluid container 26 may be configured to hold up to 300 uL of blood. The fluid container 26 may be configured to hold up to 250 uL of blood. The fluid container 26 may be configured to hold up to 200 uL of blood. The fluid container 26 may be configured to hold up to 150 uL of blood. The fluid container 26 may be configured to hold up to 100 uL of blood. The fluid container 26 may be configured to hold up to 50 uL of blood.

Referring to FIG. 4, the fluid container 26 may include a collar 67 radially extending from at least a portion thereof. The collar 67 may extend around a circumference of the fluid container 26. In some embodiments, the collar 67 may extend around a circumference of the fluid container 26. The collar 67 may be configured to receive a cap (not shown) to secure the sample within the fluid container 26 when the fluid container 26 is separated from the housing 22. The collar 67 may be a generally flat collar. In some embodiments, the collar 67 may be threaded to receive the cap.

As shown in FIGS. 10-11, in some embodiments, the puncture element 24 includes a piercing element 68. The puncture element 24 may include a group of piercing elements 68. The puncture element 24 may include a body 72. In some embodiments, the body 72 is a cylindrical shape with a proximate and a distal end. The body 72 may be made of a plastic. The body 72 may be formed around the lancet. In some embodiments, the piercing element 68 protrudes from the distal end of the body 72. The piercing element 68 may be designed for specific cut shapes, sizes, and depths. In some embodiments, the puncture element 24 is a laser or a beam of liquid. In some embodiments, the puncture element 24 travels through the fluid container 26. The blood sample may be directed by the capillary channel 62 into the fluid container 26.

In some embodiments, the disposable collection cartridge 14 includes a chemical detection element 70. The chemical detection element 70 may be capable of detecting blood characteristics during the blood collection process. The chemical detection element 70 may be capable of detecting, for example, any of red blood cells, white blood cells, platelets, neutrophils, lymphocytes, monocytes, hemoglobin and potassium. The chemical detection element 70 may be electrically coupled to the controller 46, and the controller 46 may be configured to process data transmitted by the chemical detection element 70 in real-time. In some embodiments, it may be necessary and useful to implement real-time chemical detection within the system to optimize the accuracy of the tests performed on the blood drawn during the blood draw process.

In some embodiments, the reusable actuation device 12 includes a button 19. The button 19 may be configured to initiate operation of the vacuum source 50 to prepare for actuation of the firing mechanism 78. In some embodiments, the vacuum source 50 is configured for pneumatic actuations without the use of the button 19. In some embodiments, the disposable collection cartridge 14 may have a flexible membrane 94 located between the housing 22 and the actuation button 80. In response to the vacuum source 50 providing a target pressure inside the housing 22 the flexible membrane 94 may be deformed by the pressure, such that it displaces the actuation button 80 and actuates the firing mechanism 78. In some embodiments, the flexible membrane displaces the firing mechanism 78 directly. In some embodiments, the target pressure is the pressure that is expected to produce optimal blood flow during the blood draw process.

The blood drawing device 10 described above may be used by a user for collecting a blood sample for testing. An exemplary method may include the user receiving the reusable actuation device 12 and inserting the disposable collection cartridge 14 into the cavity 20 of the reusable actuation device 12. The user may then remove the release liner covering the adhesive tape 40 on the compliant layer 32 to expose the adhesive tape 40. The user may then press the compliant layer 32 against the target area of the patient (e.g., shoulder or forearm).

Once the blood drawing device 10 has been placed in the desired location, the user may press the button 19 of the reusable actuation device 12 to initiate operation of the vacuum source 50 and/or heating element 28 to prepare the target area for actuation of the firing mechanism 78. In some embodiments, the vacuum source 50 and/or heating element 28 are configured to automatically initiate operation when the blood drawing device 10 is placed on the target area of the patient without pressing the button 19. When the desired pressure and/or temperature at the target area has been achieved, the user may press the actuation button 80 to initiate actuation of the firing mechanism 78. During collection of a sample, the vacuum source 50 and heating element 28 may be actively controlled by the controller 46 to ensure the conditions around the target area are facilitating optimal blood flow. Upon completion of the collection of the sample, the user may rotate an end retention cap 95 of the fluid container 26 to remove the fluid container. A cap may then be placed on the fluid container 26 to prevent the sample from escaping from the fluid container 26. The disposable collection cartridge 14 may then me discarded in an appropriate manner. A new disposable collection cartridge 14 may be coupled to the same reusable actuation device 12 for subsequent use on the same patient or different patients.

The term “about” or “approximately” is used herein to provide literal support for the exact number that it precedes, as well as a number that is near to or approximately the number that the term precedes. In determining whether a number is near to or approximately a specifically recited number, the near or approximating unrecited number may be a number, which, in the context in which it is presented, provides the substantial equivalent of the specifically recited number. It should be appreciated that all numerical values and ranges disclosed herein are approximate values and ranges, whether “about” is used in conjunction therewith. It should also be appreciated that the term “about,” as used herein, in conjunction with a numeral refers to a value that may be ±0.01% (inclusive), ±0.1% (inclusive), ±0.5% (inclusive), ±1% (inclusive) of that numeral, ±2% (inclusive) of that numeral, ±3% (inclusive) of that numeral, ±5% (inclusive) of that numeral, ±10% (inclusive) of that numeral, or ±15% (inclusive) of that numeral. It should further be appreciated that when a numerical range is disclosed herein, any numerical value falling within the range is also specifically disclosed.

It will be appreciated by those skilled in the art that changes could be made to the exemplary embodiments shown and described above without departing from the broad inventive concepts thereof. It is to be understood that the embodiments and claims disclosed herein are not limited in their application to the details of construction and arrangement of the components set forth in the description and illustrated in the drawings. Rather, the description and the drawings provide examples of the embodiments envisioned. The embodiments and claims disclosed herein are further capable of other embodiments and of being practiced and carried out in various ways. Specific features of the exemplary embodiments may or may not be part of the claimed invention and various features of the disclosed embodiments may be combined. Unless specifically set forth herein, the terms “a”, “an” and “the” are not limited to one element but instead should be read as meaning “at least one”. Finally, unless specifically set forth herein, a disclosed or claimed method should not be limited to the performance of their steps in the order written, and one skilled in the art can readily appreciate that the steps may be performed in any practical order.

What is claimed is:

1. A blood drawing device for collecting a blood sample from a patient, the blood drawing device comprising:
a disposable collection cartridge, the disposable collection cartridge comprising:
a housing,
a puncture element, and
a fluid container; and
a reusable actuation device releasably coupled to the disposable collection cartridge, the reusable actuation device comprising:
a controller in electronic communication with the disposable collection cartridge,
a power source in electronic communication with the disposable collection cartridge, and
a vacuum source in fluid communication with the disposable collection cartridge.

2. The blood drawing device of claim 1, wherein the disposable collection cartridge further comprises a heating element coupled to a surface of the housing extending from the reusable actuation device.

3. The blood drawing device of claim 2, wherein the heating element includes a flexible printed circuit board (PCB).

4. The blood drawing device of claim 3, wherein the heating element includes a compliant layer.

5. The blood drawing device of claim 2, wherein the heating element including an aperture, and
wherein the puncture element is movable relative to the housing and has a retracted position and an extended position, the puncture element being contained completely within the housing in the retracted position, and the puncture element extending through the aperture of the heating element in the extended position.

6. The blood drawing device of claim 5, wherein the heating element is planar and having a circular portion surrounding the aperture and a tab extending radially from the circular portion.

7. The blood drawing device of claim 6, wherein the tab includes at least one exposed electrical contact that electrically couples the heating element to the reusable actuation device.

8. The blood drawing device of claim 1, wherein the disposable collection cartridge includes an adhesive tape, and
wherein an aperture of the disposable collection cartridge includes an anticoagulation film.

9. The blood drawing device of claim 1, the reusable actuation device further comprising a controller, the controller configured to control:
the vacuum source, wherein the vacuum source is a vacuum pump;
a mechanical actuation system configured to actuate the puncture element;
a temperature sensor coupled to a heating element;
pressure sensor disposed within the disposable collection cartridge; and
a sample detection sensor disposed within the fluid container,
wherein the temperature sensor is coupled to the controller, and the controller is configured to adjust a temperature of the heating element based on a temperature detected by the temperature sensor and a desired temperature.

10. The blood drawing device of claim 9, wherein the reusable actuation device comprises an adjustable knob coupled to the controller and configured to control the desired temperature of the heating element.

11. The blood drawing device of claim 1, wherein the vacuum source is a vacuum pump configured to provide vacuum proximate a target area while the puncture element is in a retracted position and during movement of the puncture element into an extended position.

12. The blood drawing device of claim 11, wherein the vacuum pump is configured to maintain a predetermined profile of pressure in the disposable collection cartridge during collection of the blood sample.

13. The blood drawing device of claim 11, wherein the vacuum pump is configured to apply one or more variable vacuum profiles in the disposable collection cartridge during collection of the blood sample.

14. The blood drawing device of claim 1, wherein the fluid container includes a capillary channel in fluid communication with an open end of the fluid container, wherein the capillary channel includes an anticoagulation agent.

15. The blood drawing device of claim 14, wherein the fluid container includes a lip extending axially from an end portion thereof.

16. The blood drawing device of claim 1 further comprising:
a fluid reservoir, the fluid reservoir comprising a liquid reagent,
wherein the liquid reagent treats the blood during collection of the blood sample.

17. The blood drawing device of claim 16, wherein the liquid reagent comprises at least one of lithium heparin, K2-EDTA, K3-EDTA, trisodium citrate or another acceptable testing or anticoagulant reagent.

18. The blood drawing device of claim 1, wherein the puncture element includes one or more lancets.

19. The blood drawing device of claim 1, wherein the vacuum source is a vacuum pump configured to continuously pump air out of the disposable collection cartridge before and during collection of the blood sample.

20. The blood drawing device of claim 1, wherein the fluid container is coupled to the disposable collection cartridge by a threaded coupling.

21. The blood drawing device of claim 1, wherein the fluid container is positioned at an oblique angle relative to the puncture element when the disposable collection cartridge is coupled with the reusable actuation device.

22. A blood drawing device for collecting a blood sample from a patient, the blood drawing device comprising:
a disposable collection cartridge, the disposable collection cartridge comprising:
a housing,
a puncture element movable relative to the housing,
a heating element, and
at least one fluid container; and
a reusable actuation device releasably coupled to the disposable collection cartridge, the reusable actuation device comprising:
a vacuum pump fluidly coupled to the disposable collection cartridge and configured to continuously pump air out of the disposable collection cartridge before and during collection of the blood sample,
a controller in electronic communication with the disposable collection cartridge, and
a power source in electronic communication with the disposable collection cartridge,
wherein the vacuum pump is configured to maintain a predetermined profile of pressure in the disposable collection cartridge during collection of the blood sample,
wherein the heating element includes a flexible printed circuit board (PCB) and a compliant layer,
wherein the controller is electrically coupled to the heating element and a temperature sensor is coupled to the heating element to adjust a temperature of the heating element, and
wherein the heating element includes an aperture, and the puncture element has a retracted position and an extended position, the puncture element being contained completely within the housing in the retracted position, and the puncture element extending through the aperture of the heating element in the extended position.
